(19) United States
(12) Patent Application Publication
(10) Pub. No.: US 2026/0160733 A1
(43) Pub. Date: Jun. 11, 2026

(54) ULTRASOUND IMAGE PROCESSING APPARATUS AND ULTRASOUND IMAGE PROCESSING PROGRAM

(71) Applicant: FUJIFILM Corporation, Tokyo (JP)

(72) Inventor: Ryo UMEMURA, Tokyo (JP)

(73) Assignee: FUJIFILM Corporation, Tokyo (JP)

(21) Appl. No.: 19/391,984

(22) Filed: Nov. 17, 2025

(30) Foreign Application Priority Data
Dec. 11, 2024 (JP) 2024-216999

(51) Int. Cl. G01N 29/06; G01N 29/44

(52) U.S. Cl. CPC G01N 29/0645; G01N 29/0672; G01N 29/44

(57) ABSTRACT

An ultrasound diagnostic apparatus includes a trackball as a pointing device. A parameter setting unit changes a value of a main parameter “dynamic range” as a first parameter, in a case where the trackball is operated in a first axial direction, and changes a value of a main parameter “gain” as a second parameter, in a case where the trackball is operated in a second axial direction.

### CROSS-REFERENCE TO RELATED APPLICATION

This application claims the priority benefit of Japan application serial no. 2024-216999, filed on Dec. 11, 2024. The entirety of the above-mentioned patent application is hereby incorporated by reference herein and made a part of this specification.

### BACKGROUND OF THE INVENTION

### 1. Field of the Invention

The present specification discloses improvements in an ultrasound image processing apparatus and an ultrasound image processing program.

### 2. Description of the Related Art

Conventionally, an ultrasound diagnostic apparatus is known that transmits an ultrasound wave to a subject, acquires a reception signal based on a reflected wave from the subject, and forms an ultrasound image based on the reception signal. In addition, an ultrasound image processing apparatus is known that executes various kinds of processing relating to the ultrasound image (referred to as “ultrasound image processing” in the present specification). The ultrasound image processing apparatus may be the ultrasound diagnostic apparatus itself, or another apparatus, for example, a server computer or the like.

The concept of ultrasound image processing in the present specification may include all kinds of processing related to ultrasound images. The ultrasound image processing includes, for example, transmission processing of ultrasound waves, signal processing for a reception signal, image formation processing of forming an ultrasound image from the reception signal, correction processing for the ultrasound image, display processing of the ultrasound image, and the like.

An ultrasound image processing apparatus is generally provided with an input interface through which an operator inputs various instructions. The input interface may include, in addition to buttons, knobs, a mouse, a keyboard, and the like, a pointing device capable of scanning in a plurality of directions. The operator can change, for example, a value of a parameter related to the ultrasound image processing by using the pointing device.

Conventionally, techniques related to an operation of the pointing device by an operator have been proposed. For example, JP2002-238892A discloses an ultrasound diagnostic apparatus comprising a trackball as a pointing device, a parameter selection unit that selects a type of parameter in accordance with the trackball being rotated in a longitudinal direction, and a parameter adjustment unit that changes a value of the selected type of parameter in accordance with the trackball being rotated in a lateral direction.

### SUMMARY OF THE INVENTION

Meanwhile, there are cases where an operator of the ultrasound image processing apparatus may desire to change values of a plurality of parameters related to the ultrasound image processing. In such a case, conventionally, the operator performs an operation such as selecting the type of parameter to be changed by operating a button or the like, changing the value of the parameter by operating a trackball or the like, then again selecting the type of parameter to be changed by operating the button or the like, and changing the value of the parameter by operating the trackball or the like.

Since such an operation is complicated for the operator, values of a plurality of parameters related to ultrasound image processing may preferably be easily changeable by the operator. In particular, in a case where the ultrasound image processing apparatus is an ultrasound diagnostic apparatus, the operator (an operator of the ultrasound diagnostic apparatus) holds an ultrasound probe with one hand while operating the input interface with the other hand (that is, with a single hand). Accordingly, in a case where the ultrasound image processing apparatus is an ultrasound diagnostic apparatus, it is more advantageous that values of the plurality of parameters related to ultrasound image processing can be easily changed.

An object of the ultrasound image processing apparatus disclosed in the present specification is to enable an operator to easily change values of a plurality of parameters related to ultrasound image processing.

An ultrasound image processing apparatus disclosed in the present specification is an ultrasound image processing apparatus comprising: a processor; and a pointing device that is operable by an operator in a plurality of directions, in which the processor is configured to: in a case where the pointing device is operated in a first axial direction, change a value of a first parameter, which is a parameter related to ultrasound image processing that is processing relating to an ultrasound image formed by transmitting and receiving an ultrasound wave with respect to a subject; and in a case where the pointing device is operated in a second axial direction different from the first axial direction, change a value of a second parameter, which is a parameter related to the ultrasound image processing and different from the first parameter.

The processor may preferably be configured to: change the values of the first parameter and the second parameter in a case where the pointing device is operated in a third axial direction that is a direction between the first axial direction and the second axial direction.

The pointing device may preferably be configured to enable the operator to input an operation amount in each direction, and the processor may preferably be configured to: determine a change amount of the value of the first parameter based on a component in the first axial direction of an operation amount in the third axial direction; and determine a change amount of the value of the second parameter based on a component in the second axial direction of the operation amount in the third axial direction.

The parameter may preferably include a main parameter and a plurality of sub-parameters for determining a value of the main parameter, the ultrasound image processing apparatus may preferably be operable in a first operation mode in which values of a plurality of the main parameters are changeable, and a second operation mode in which values of the plurality of sub-parameters are changeable, and the processor may preferably be configured to: in a case where the pointing device is operated in the first axial direction while operating in the second operation mode, change a value of a first sub-parameter, which is one of the plurality of sub-parameters; and in a case where the pointing device is operated in the second axial direction, change a value of a second sub-parameter, which is a sub-parameter different from the first sub-parameter.

The processor may preferably be configured to: cause a display unit to display a characteristic index indicating characteristics of an ultrasound image processed using the changed first parameter and the changed second parameter.

In addition, an ultrasound image processing program disclosed in the present specification is an ultrasound image processing program for causing a computer including a pointing device that is operable by an operator in a plurality of directions to execute: in a case where the pointing device is operated in a first axial direction, changing a value of a first parameter, which is a parameter related to ultrasound image processing that is processing relating to an ultrasound image formed by transmitting and receiving an ultrasound wave with respect to a subject; and in a case where the pointing device is operated in a second axial direction different from the first axial direction, changing a value of a second parameter, which is a parameter related to the ultrasound image processing and different from the first parameter.

With the ultrasound image processing apparatus disclosed in the present specification, the operator can easily change values of a plurality of parameters related to ultrasound image processing.

### BRIEF DESCRIPTION OF THE DRAWINGS

FIG. 1 is a schematic diagram of a configuration of an ultrasound diagnostic apparatus according to the present embodiment.

FIG. 2 is a plan view of a trackball.

FIG. 3 is a transition diagram of an operation mode of the ultrasound diagnostic apparatus.

FIG. 4 is a diagram showing an example of a main parameter setting screen.

FIG. 5A is a diagram showing a state in which the trackball is operated in a −X-axis direction.

FIG. 5B is a diagram showing a state in which a value of a dynamic range has been changed on the main parameter setting screen.

FIG. 6A is a diagram showing a state in which the trackball is operated in a +Y-axis direction.

FIG. 6B is a diagram showing a state in which a value of a gain has been changed on the main parameter setting screen.

FIG. 7A is a diagram showing a state in which the trackball is operated in a −Y-axis direction while an operation button is being pressed.

FIG. 7B is a diagram showing a state in which a value of low echo reduction has been changed on the main parameter setting screen.

FIG. 8 is a diagram showing a correspondence relationship between an operation direction of the trackball and presence or absence of an operation of an operation button, and a main parameter to be changed for each parameter type.

FIG. 9A is a diagram showing a state in which the trackball is operated in an oblique direction between the −X-axis direction and the +Y-axis direction.

FIG. 9B is a diagram showing a state in which the values of the gain and the dynamic range have been changed on the main parameter setting screen.

FIG. 10 is a diagram showing an operation amount of the trackball in the oblique direction and a component in an X-axis direction and a component in a Y-axis direction of the operation amount.

FIG. 11 is a diagram showing examples of a main parameter and sub-parameters.

FIG. 12 is a diagram showing an example of a sub-parameter setting screen.

FIG. 13 is a diagram showing another example of an image as a characteristic index.

FIG. 14A is a diagram showing a state in which the trackball is operated in a +X-axis direction.

FIG. 14B is a diagram showing a state in which a value of noise reduction has been changed on the sub-parameter setting screen.

FIG. 15A is a diagram showing a state in which the trackball is operated in the +Y-axis direction.

FIG. 15B is a diagram showing a state in which a value of edge enhancement has been changed on the sub-parameter setting screen.

FIG. 16 is a diagram showing a correspondence relationship between the operation direction of the trackball and the presence or absence of the operation of the operation button, and a sub-parameter to be changed for each main parameter.

FIG. 17 is a diagram showing an example of a registration screen of the sub-parameter.

### DESCRIPTION OF THE PREFERRED EMBODIMENTS

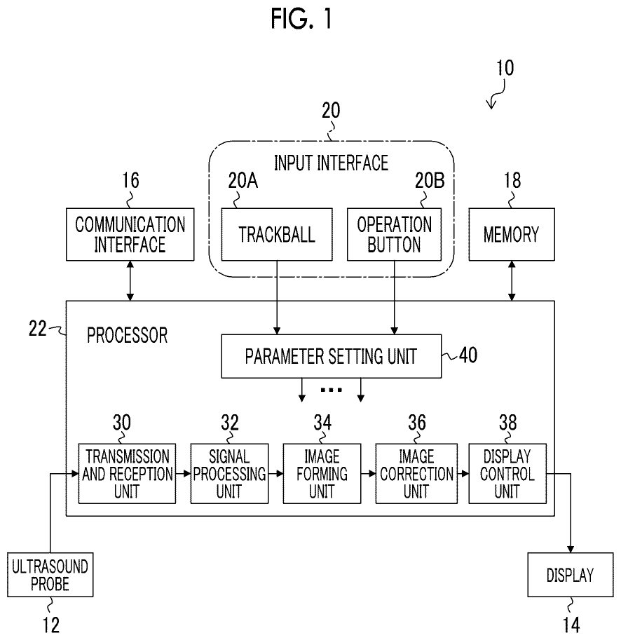

FIG. 1 is a schematic diagram of a configuration of an ultrasound diagnostic apparatus 10 as an ultrasound image processing apparatus according to the present embodiment. The ultrasound diagnostic apparatus 10 is a medical apparatus installed in medical institutions, such as a hospital. Although details will be described below, the ultrasound diagnostic apparatus 10 is an apparatus that transmits ultrasound waves to a subject, acquires reception signals based on reflected waves of the ultrasound waves from the subject, forms an ultrasound image based on the reception signals, and displays the ultrasound image on a display unit. That is, the ultrasound diagnostic apparatus 10 is an apparatus that executes the ultrasound image processing. The ultrasound image includes, for example, an ultrasound tomographic image (B-mode image), an M-mode image representing a motion of each site of the subject on a transmission beam, a Doppler image representing a flow rate and a flow direction of blood or the like, and the like.

The ultrasound diagnostic apparatus 10 includes an ultrasound probe 12, a display 14, a communication interface 16, a memory 18, an input interface 20, and a processor 22.

The ultrasound probe 12 is a device that transmits and receives ultrasound waves with respect to the subject. The ultrasound probe 12 includes a transducer element array consisting of a plurality of transducer elements. The transducer element array transmits ultrasound waves to the subject based on a transmission signal, which is an electrical signal supplied from the processor 22 to be described below. Additionally, the transducer element array receives a reflected wave from the subject, converts the reflected wave into a reception signal, which is an electrical signal, and outputs the reception signal to the processor 22.

The display 14 as a display unit is a display device configured using, for example, a liquid crystal display, an organic electro luminescence (EL), or the like. The display 14 displays various screens in accordance with an instruction from the processor 22.

The communication interface 16 is configured using, for example, a network adapter or the like. The communication interface 16 exerts a function of communicating with another device via a communication line such as a wide area network (WAN) or a local area network (LAN).

A memory 18 includes a hard disk drive (HDD), a solid state drive (SSD), an embedded multi media card (eMMC), a read only memory (ROM), a random access memory (RAM), or the like. The memory 18 stores an ultrasound image processing program for operating each unit of the ultrasound diagnostic apparatus 10. The ultrasound image processing program can also be stored in, for example, a non-transitory computer-readable storage medium such as a universal serial bus (USB) memory or a CD-ROM. The ultrasound diagnostic apparatus 10 can read the ultrasound image processing program from the storage medium and execute the ultrasound image processing program.

The input interface 20 is used to input a command of an operator of the ultrasound diagnostic apparatus 10 as an operator to the ultrasound diagnostic apparatus 10. The input interface 20 includes a pointing device. The pointing device is a device that can be operated by the operator in a plurality of directions (at least two directions). Preferably, the pointing device may be a device with which the operator can also input an operation amount in each direction. In the present embodiment, the input interface 20 includes a trackball 20A as a pointing device. The pointing device is not limited to the trackball 20A and may be, for example, a joystick, a pointing stick, a touch pad, or the like.

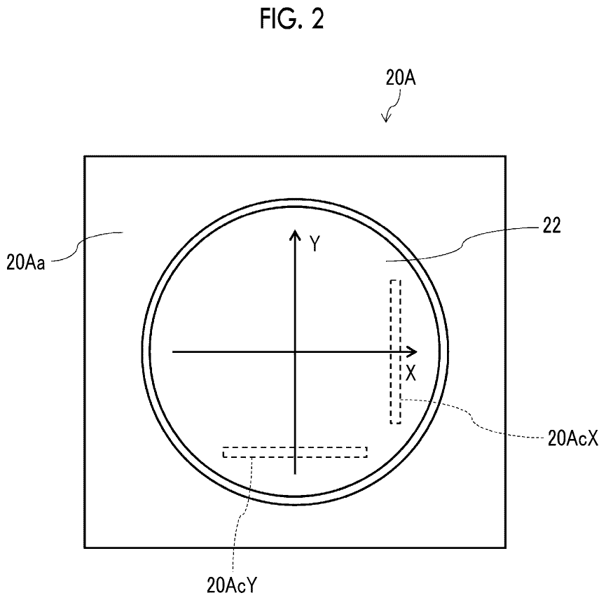

FIG. 2 is a plan view of the trackball 20A. The trackball 20A includes a base portion 20Aa, a ball portion 20Ab having a spherical shape, and sensors 20AcX and 20AcY. The base portion 20Aa includes a circular opening portion in plan view, and the ball portion 20Ab is provided such that a part of the ball portion 20Ab is exposed to an outside through the opening portion. The operator operates the trackball 20A by rotating the ball portion 20Ab. Since the ball portion 20Ab has a spherical shape, the operator can rotate the ball portion 20Ab in any direction within 360 degrees in plan view. That is, the trackball 20A can be operated by the operator in a plurality of directions. In the present specification, as shown in FIG. 2, a rotation direction of the ball portion 20Ab will be referred to as an X-axis direction as a first axial direction in a lateral direction, and a Y-axis direction as a second axial direction in a longitudinal direction. Additionally, in the present specification, a direction between the X-axis direction and the Y-axis direction will be referred to as an oblique direction as a third axial direction.

In particular, in the present specification, in a case where the ball portion 20Ab is rotated to a right side along the X-axis direction, it is expressed that the trackball 20A is operated in a +X-axis direction, in a case where the ball portion 20Ab is rotated to a left side along the X-axis direction, it is expressed that the trackball 20A is operated in a −X-axis direction, in a case where the ball portion 20Ab is rotated to an upper side along the Y-axis direction, it is expressed that the trackball 20A is operated in a +Y-axis direction, and in a case where the ball portion 20Ab is rotated to a lower side along the Y-axis direction, it is expressed that the trackball 20A is operated in a −Y-axis direction. In a case of simply referring to the X-axis direction, it is a concept encompassing the +X-axis direction and the −X-axis direction, and in a case of simply referring to the Y-axis direction, it is a concept encompassing the +Y-axis direction and the −Y-axis direction.

The sensors 20AcX and 20AcY include, for example, an optical sensor or a rotary encoder. The sensors 20AcX and 20AcY are sensors that detect a rotation direction and a rotation amount of the ball portion 20Ab. The sensor 20AcX detects the rotation in the X-axis direction (lateral direction) and the rotation amount of the ball portion 20Ab, and the sensor 20AcY detects the rotation in the Y-axis direction (longitudinal direction) and the rotation amount of the ball portion 20Ab. In a case where the ball portion 20Ab is operated in an oblique direction between the X-axis direction and the Y-axis direction, the rotation direction in the oblique direction and the rotation amount can be detected based on the rotation amounts detected by both the sensor 20AcX and the sensor 20AcY (in a manner of vector synthesis).

Description returns to FIG. 1. The input interface 20 includes an operation button 20B. The operation button 20B may be any button as long as presence or absence of an operation from the operator can be identified. The operation button 20B may be, for example, a mechanical button, a specific key of a keyboard, or a touch panel. The input interface 20 may include a plurality of operation buttons 20B.

The processor 22 exerts functions as a transmission and reception unit 30, a signal processing unit 32, an image forming unit 34, an image correction unit 36, a display control unit 38, and a parameter setting unit 40 by the ultrasound image processing program stored in the memory 18.

The transmission and reception unit 30 transmits a transmission signal to the ultrasound probe 12 (specifically, each transducer element of the transducer element array). The transmission signal may include a plurality of parameters for determining a transmission intensity or a transmission frequency of ultrasound waves. These parameters are set by the parameter setting unit 40 (which will be described below). In addition, the transmission and reception unit 30 receives, from each transducer element, a reception signal based on the reflected wave from the subject. The transmission and reception unit 30 performs reception beamforming processing including AD conversion processing on the reception signal, thinning processing of digital reception signals, phase alignment and addition processing in which phases of reception signals from the respective transducer elements are aligned and added, and the like. In the reception beamforming processing, a plurality of parameters set by the parameter setting unit 40 may also be used. By the reception beamforming processing, a reception beam signal is formed in which information indicating the signal intensity of the reflected wave from the subject is arranged in a depth direction of the subject. In the reception beamforming processing, a plurality of parameters set by the parameter setting unit 40 may also be used.

The signal processing unit 32 executes various kinds of signal processing including filter processing of applying a bandpass filter, detection processing, and the like on the reception beam signal from the transmission and reception unit 30. Additionally, the signal processing unit 32 may also obtain a Doppler signal indicating a flow rate and a flow direction of a blood flow or the like in the subject by executing quadrature detection processing, autocorrelation calculation, or the like, on the reception beam signal. In the signal processing by the signal processing unit 32, the plurality of parameters set by the parameter setting unit 40 may be used.

The image forming unit 34 forms an ultrasound image based on the reception beam signal subjected to signal processing by the signal processing unit 32. As mentioned above, the ultrasound image may include a B-mode image, an M-mode image, a Doppler image, and the like. In the image formation processing by the image forming unit 34, the plurality of parameters set by the parameter setting unit 40 may also be used.

The image correction unit 36 performs correction processing on the ultrasound image formed by the image forming unit 34. For example, the image correction unit 36 performs correction processing such as grayscale correction, contrast correction, and image quality correction. In the correction processing by the image correction unit 36, the plurality of parameters set by the parameter setting unit 40 may also be used.

The display control unit 38 performs display processing of displaying various images including the ultrasound tomographic image formed by the image forming unit 34 on the display 14. The display processing of the display control unit 38 also includes processing of changing the setting (particularly, the display setting) of the display 14. In the display processing by the display control unit 38, the plurality of parameters set by the parameter setting unit 40 may also be used.

The parameter setting unit 40 changes the values of the parameters related to the various kinds of ultrasound image processing mentioned above in accordance with an instruction from the operator input through the input interface 20. Before describing details of processing of the parameter setting unit 40, a hierarchical structure of parameters and a plurality of operation modes of the ultrasound diagnostic apparatus 10 will be described.

In the present embodiment, the parameters include a main parameter and a plurality of sub-parameters for determining a value of the main parameter. The sub-parameters are positioned subordinate to the main parameter, and in this sense, the parameters in the present embodiment have a hierarchical structure. Although details will be described below, for example, a value of a main parameter “resolution” is composed of a value of a sub-parameter “noise reduction” and a value of a sub-parameter “edge enhancement”. In the present specification, in a case of simply referring to “parameter”, it is a concept encompassing the main parameter and the sub-parameters.

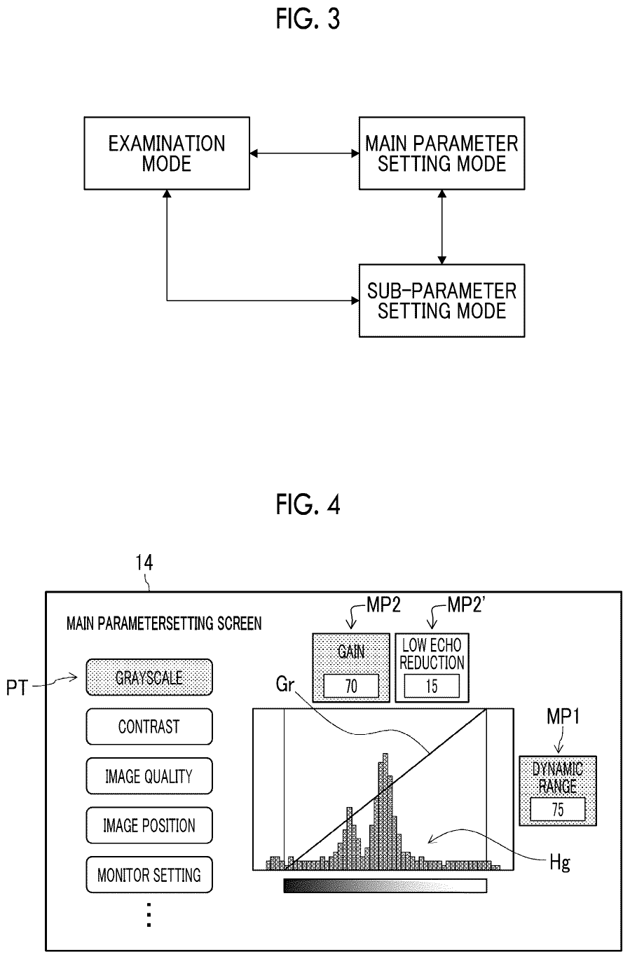

Next, the plurality of operation modes of the ultrasound diagnostic apparatus 10 will be described. FIG. 3 is a transition diagram of the operation mode of the ultrasound diagnostic apparatus 10. An examination mode is a mode in which an ultrasound image is formed and displayed by transmitting and receiving ultrasound waves with respect to the subject. In a case where a transition instruction to a main parameter setting mode is received from the operator in the examination mode, the ultrasound diagnostic apparatus 10 transitions to the main parameter setting mode as a first operation mode. The main parameter setting mode is an operation mode in which the value of the main parameter can be changed. In the present embodiment, a plurality of main parameters are classified into a plurality of parameter types according to the contents thereof. For example, the main parameter “gain”, the main parameter “dynamic range”, and the main parameter “low echo reduction” are classified into a parameter type “grayscale”, and the main parameter “resolution” and the main parameter “texture” are classified into a parameter type “image quality”. In the main parameter setting mode, a main parameter setting screen as shown in FIG. 4 (details will be described below) is displayed on the display 14.

In addition, in a case where a transition instruction to a sub-parameter setting mode is received from the operator in the examination mode, the ultrasound diagnostic apparatus 10 transitions to the sub-parameter setting mode as a second operation mode. The sub-parameter setting mode is an operation mode in which the value of the sub-parameter can be changed. The transition to the sub-parameter setting mode (that is, changing the values of the sub-parameters) may be allowed to be performed only by an administrator of the ultrasound image processing apparatus (the ultrasound diagnostic apparatus 10 in the present embodiment), or may also be allowed to be performed by an operator other than the administrator. In the sub-parameter setting mode, a sub-parameter setting screen as shown in FIG. 12 (details will be described below) is displayed on the display 14. Note that a transition from the main parameter setting mode directly to the sub-parameter setting mode may be possible, and a transition from the sub-parameter setting mode directly to the main parameter setting mode may also be possible.

Hereinafter, details of the setting processing of the value of the main parameter will be described with reference to FIGS. 4 to 10. In a case where the ultrasound diagnostic apparatus 10 transitions to the main parameter setting mode, the display control unit 38 displays the main parameter setting screen as shown in FIG. 4 on the display 14. On the main parameter setting screen, a plurality of parameter types PT are displayed. The operator selects a parameter type PT including the main parameter to be changed, from among the plurality of parameter types PT. Here, it is assumed that a parameter type “grayscale” is selected.

In a case where the parameter type PT is selected by the operator, the display control unit 38 displays items and values of a plurality of main parameters included in the selected parameter type PT on the display 14. In the example of FIG. 4, the display control unit 38 causes the display 14 to display an item and a value of a main parameter “dynamic range” as a first parameter indicated by reference numeral MP1, an item and a value of a main parameter “gain” as a second parameter indicated by reference numeral MP2, and an item and a value of a main parameter “low echo reduction” as another second parameter indicated by reference numeral MP2′.

Preferably, in a case where the parameter type PT is selected by the operator, the display control unit 38 may cause the display 14 to display, as characteristics of an ultrasound image formed immediately before entering the main parameter setting mode (here, a B-mode image, hereinafter simply referred to as an “ultrasound image”), a characteristic index indicating characteristics related to a plurality of main parameters included in the selected parameter type PT. In the example of FIG. 4, since the parameter type “grayscale” including the main parameter “dynamic range”, the main parameter “gain”, and the main parameter “low echo reduction” is selected, the display control unit 38 displays a histogram Hg and a graph Gr as the characteristic index. The histogram Hg is a histogram of the signal intensity of the reception beam signal, in which a horizontal axis represents the signal intensity (the farther to the right, the greater the signal intensity), and a vertical axis represents the frequency. The graph Gr represents a correspondence relationship between each signal intensity represented by the histogram Hg and a brightness value of the ultrasound image, in which a horizontal axis represents the signal intensity (the farther to the right, the greater the signal intensity), and a vertical axis represents the brightness value. A signal intensity at a left end of the graph Gr represents a signal intensity corresponding to a minimum brightness value, and a signal intensity at a right end of the graph Gr represents a signal intensity corresponding to a maximum brightness value.

On the main parameter setting screen, the parameter setting unit 40 changes the values of the plurality of main parameters included in the selected parameter type PT in response to the operation of the trackball 20A by the operator. Specifically, the parameter setting unit 40 determines, according to the operation direction of the trackball 20A, a main parameter whose value is to be changed from among the plurality of main parameters. In the present embodiment, in a case where the trackball 20A is operated in the first axial direction (the X-axis direction (refer to FIG. 2), that is, the lateral direction), the parameter setting unit 40 changes the value of the main parameter “dynamic range” as the first parameter, and in a case where the trackball 20A is operated in the second axial direction (the Y-axis direction (refer to FIG. 2), that is, the longitudinal direction), the parameter setting unit 40 changes the value of the main parameter “gain” as the second parameter.

In a case where the trackball 20A is operated in the X-axis direction (lateral direction), the value of the main parameter “dynamic range” is changed, and in a case where the trackball 20A is operated in the Y-axis direction (longitudinal direction), the value of the main parameter “gain” (or the main parameter “low echo reduction” as will be described below) is changed. Accordingly, the display control unit 38 may preferably display an item and a value of the main parameter “dynamic range” and items and values of the main parameters “gain” and “low echo reduction” such that the operator can easily visualize which main parameter value will change depending on the direction in which the trackball 20A is operated. For example, as shown in FIG. 4, the display control unit 38 may preferably display, on a left side or a right side of the characteristic index (the histogram Hg and the graph Gr), the main parameter “dynamic range” whose value is changed in a case where the trackball 20A is operated in the X-axis direction (lateral direction), and display, on an upper side or a lower side of the characteristic index, the main parameters “gain” and “low echo reduction” whose values are changed in a case where the trackball 20A is operated in the Y-axis direction (longitudinal direction).

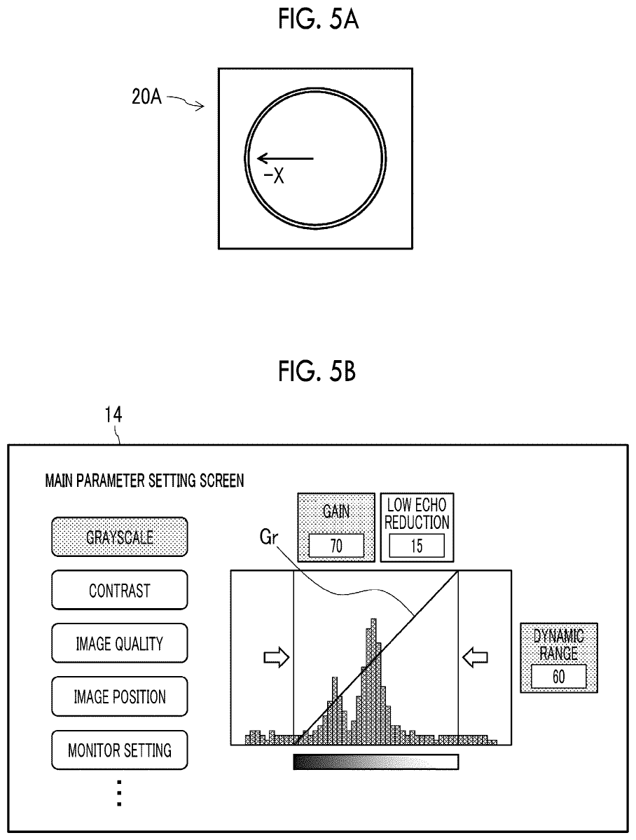

FIG. 5A is a diagram showing a state in which the trackball 20A is operated in the −X-axis direction. FIG. 5B is a diagram showing a state in which the value of the main parameter “dynamic range” as the first parameter has been changed on the main parameter setting screen in response to the operation of the trackball 20A in the −X-axis direction. As shown in FIG. 5A, in a case where the trackball 20A is operated in the −X-axis direction, the parameter setting unit 40 decreases the value of the main parameter “dynamic range”. As mentioned above, since the trackball 20A can detect the operation amount (the rotation amount of the ball portion 20Ab), the parameter setting unit 40 may preferably determine the change amount of the value of the main parameter “dynamic range” in accordance with the operation amount of the trackball 20A in the −X-axis direction.

In a case where the value of the main parameter “dynamic range” decreases, the display control unit 38 changes the graph Gr on the main parameter setting screen such that a left end of the graph Gr moves to the right side (a greater signal intensity side), a right end of the graph Gr moves to the left side (a smaller signal intensity side), and a slope of the graph Gr is greater. The changed graph Gr indicates characteristics of the ultrasound image processed using the changed value of the main parameter “dynamic range”. That is, the display control unit 38 may preferably cause the display unit to display the characteristic index indicating the characteristics of the ultrasound image processed using the changed main parameter. As a result, the operator can easily understand the characteristics of the ultrasound image after the main parameter has been changed.

On the contrary, in a case where the trackball 20A is operated in the +X-axis direction, the parameter setting unit 40 increases the value of the main parameter “dynamic range”. Here, the parameter setting unit 40 may preferably determine the change amount of the value of the main parameter “dynamic range” in accordance with the operation amount of the trackball 20A in the +X-axis direction.

In a case where the value of the main parameter “dynamic range” increases, the display control unit 38 changes the graph Gr on the main parameter setting screen such that the left end of the graph Gr moves to the left side (the smaller signal intensity side), the right end of the graph Gr moves to the right side (the greater signal intensity side), and the slope of the graph Gr is smaller.

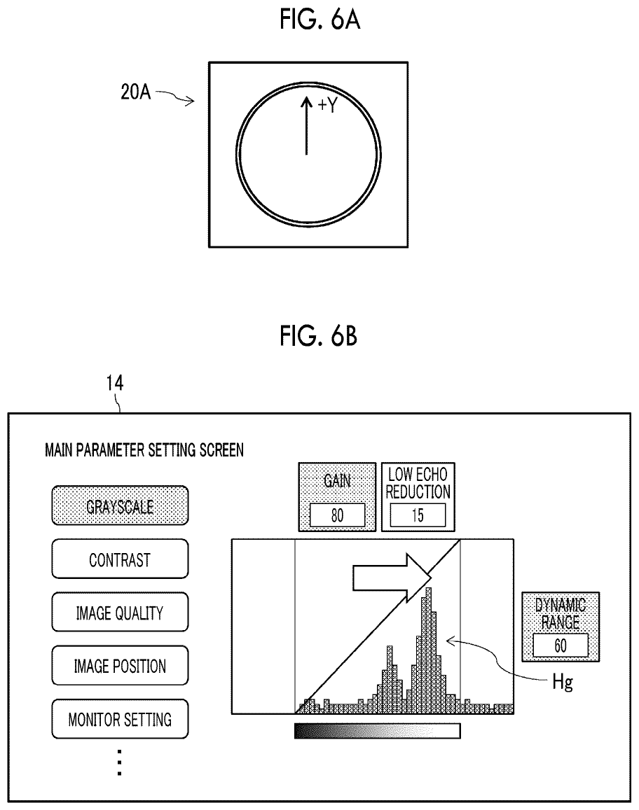

FIG. 6A is a diagram showing a state in which the trackball 20A is operated in the +Y-axis direction. FIG. 6B is a diagram showing a state in which the value of the main parameter “gain” as the second parameter has been changed on the main parameter setting screen in response to the operation of the trackball 20A in the +Y-axis direction. As shown in FIG. 6A, in a case where the trackball 20A is operated in the +Y-axis direction, the parameter setting unit 40 increases the value of the main parameter “gain”. Here, the parameter setting unit 40 may preferably determine the change amount of the value of the main parameter “gain” in accordance with the operation amount of the trackball 20A in the +Y-axis direction.

In a case where the value of the main parameter “gain” increases, the display control unit 38 changes the histogram Hg on the main parameter setting screen such that the histogram Hg moves to the right side (a greater signal intensity side) as a whole. The changed histogram Hg indicates the characteristics of the ultrasound image processed using the changed value of the main parameter “gain”.

On the contrary, in a case where the trackball 20A is operated in the −Y-axis direction, the parameter setting unit 40 decreases the value of the main parameter “gain”. Here, the parameter setting unit 40 may preferably determine the change amount of the value of the main parameter “gain” in accordance with the operation amount of the trackball 20A in the −Y-axis direction.

In a case where the value of the main parameter “gain” decreases, the display control unit 38 changes the histogram Hg on the main parameter setting screen such that the histogram Hg moves to the left side (the smaller signal intensity side) as a whole.

In this manner, according to the present embodiment, the operator can change the values of the two main parameters only by operating the trackball 20A. Accordingly, the operator can easily change the values of the plurality of main parameters related to the ultrasound image processing as compared with the conventional case.

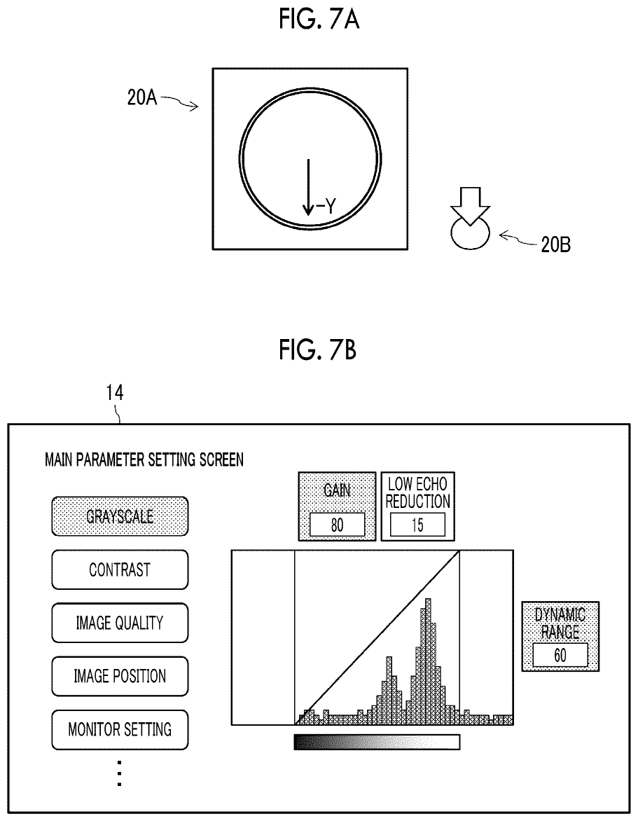

The parameter setting unit 40 may preferably switch the main parameter whose value is to be changed in a case where the trackball 20A is operated in response to the operation of the operation button 20B. FIG. 7A is a diagram showing a state in which the trackball 20A is operated in the −Y-axis direction while the operation button 20B is being pressed. FIG. 7B is a diagram showing a state in which the value of the main parameter “low echo reduction” as the second parameter has been changed on the main parameter setting screen in response to the operation of the trackball 20A in the −Y-axis direction while the operation button 20B is being pressed. As shown in FIG. 7A, in a case where the operation button 20B is pressed, the parameter setting unit 40 switches, from “gain” to “low echo reduction”, the main parameter whose value is to be changed in a case where the trackball 20A is operated in the Y-axis direction. In the present embodiment, the main parameter whose value is to be changed in a case where the trackball 20A is operated in the Y-axis direction while the operation button 20B is being pressed is “low echo reduction”. However, in response to the operation button 20B being pressed, the main parameter whose value is to be changed in a case where the trackball 20A is operated in the Y-axis direction may be switched in a toggle manner between “gain” and “low echo reduction”.

The display control unit 38 may preferably explicitly indicate, on the main parameter setting screen, which main parameter is to have its value changed by the operation of the trackball 20A. For example, as shown in FIG. 7B, in a case where the operation button 20B is pressed, the display control unit 38 may preferably highlight (in the example of FIG. 7B, by hatching) “low echo reduction” which is a main parameter whose value is to be changed by the operation of the trackball 20A.

In a case where the trackball 20A is operated in the −Y-axis direction in a state in which the operation button 20B is pressed, the parameter setting unit 40 decreases the value of the main parameter “low echo reduction”. On the contrary, in a case where the trackball 20A is operated in the +Y-axis direction in a state in which the operation button 20B is pressed, the parameter setting unit 40 increases the value of the main parameter “low echo reduction”. Here, the parameter setting unit 40 may preferably determine the change amount of the value of the main parameter “low echo reduction” in accordance with the operation amount of the trackball 20A in the Y-axis direction.

In the examples of FIGS. 7A and 7B, in a case where the operation button 20B is pressed, the main parameter whose value is to be changed in a case where the trackball 20A is operated in the Y-axis direction is switched. Instead of or in addition to this, in a case where the operation button 20B is pressed, the main parameter whose value is to be changed in a case where the trackball 20A is operated in the X-axis direction may be switched. In addition, in a case where there are a plurality of operation buttons 20B, the parameter setting unit 40 may select a main parameter whose value is to be changed in a case where the trackball 20A is operated in the Y-axis direction, in accordance with the pressed operation button 20B. For example, in a case where the trackball 20A is operated in the Y-axis direction without operating the operation button 20B, a value of a first main parameter is changed, in a case where the trackball 20A is operated in the Y-axis direction while a first operation button 20B is being pressed, a value of a second main parameter is changed, and in a case where the trackball 20A is operated in the Y-axis direction while a second operation button 20B is being pressed, a value of a third main parameter is changed.

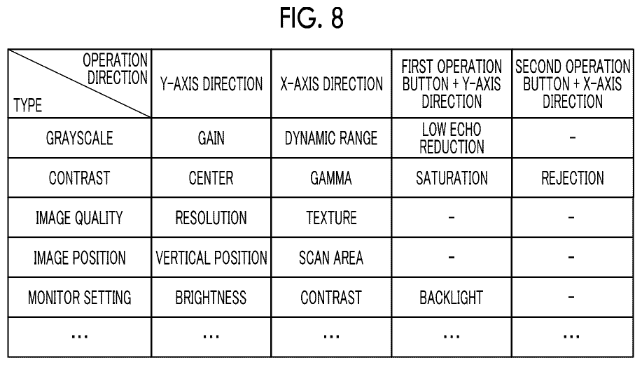

Which main parameter is to be changed in a case where the trackball 20A is operated in each of the X-axis direction and the Y-axis direction or in a case where the trackball 20A is operated in each of the X-axis direction and the Y-axis direction while the operation button 20B is being pressed is set in advance. FIG. 8 is a diagram showing a correspondence relationship between the operation direction of the trackball 20A and the presence or absence of the operation of the operation button 20B, and the main parameter to be changed for each parameter type PT.

In the table shown in FIG. 8, each row corresponds to each parameter type PT, and each column corresponds to the operation content of the input interface 20. For example, with respect to the parameter type “grayscale”, the main parameter “gain” is set for the operation of the trackball 20A in the Y-axis direction, the main parameter “dynamic range” is set for the operation of the trackball 20A in the X-axis direction, and the main parameter “low echo reduction” is set for the operation of the trackball 20A in the Y-axis direction and the first operation button 20B. In addition, with respect to the parameter type “contrast”, the main parameter “center” is set for the operation of the trackball 20A in the Y-axis direction, the main parameter “gamma” is set for the operation of the trackball 20A in the X-axis direction, the main parameter “saturation” is set for the operation of the trackball 20A in the Y-axis direction and the first operation button 20B, and the main parameter “rejection” is set for the operation of the trackball 20A in the X-axis direction and the second operation button 20B.

Here, a parameter suggestive of “wide”, “narrow”, or the like (for example, the main parameter “dynamic range”) may preferably be set for the operation of the trackball 20A in the X-axis direction (lateral direction), and a parameter suggestive of “high”, “low”, or the like (for example, the main parameter “gain”) may preferably be set for the operation of the trackball 20A in the Y-axis direction (longitudinal direction). As a result, the operator can more intuitively change the value of the target main parameter by using the trackball 20A.

Additionally, in the example of FIG. 8, the main parameter is set for the operation of the first operation button 20B and the trackball 20A in the Y-axis direction, but the main parameter is not set for the operation of the first operation button 20B and the trackball 20A in the X-axis direction. Similarly, the main parameter is set for the operation of the second operation button 20B and the trackball 20A in the X-axis direction, but the main parameter is not set for the operation of the second operation button 20B and the trackball 20A in the Y-axis direction. This is devised to reduce erroneous operations by the operator as much as possible, since operations combining the trackball 20A and the operation button 20B are slightly complicated.

The table as shown in FIG. 8 is stored in the memory 18. The parameter setting unit 40 determines the main parameter whose value is to be changed in a case where the trackball 20A is operated, or in a case where the operation button 20B and the trackball 20A are operated, in accordance with the parameter type PT selected by the operator, while referring to the table.

As mentioned above, since the trackball 20A can be operated in any direction within 360 degrees in plan view, the trackball 20A can be operated in the oblique direction. In a case where the trackball 20A is operated in the oblique direction, the parameter setting unit 40 may preferably change both the value of the main parameter “dynamic range” as the first parameter and the value of the main parameter “gain” as the second parameter.

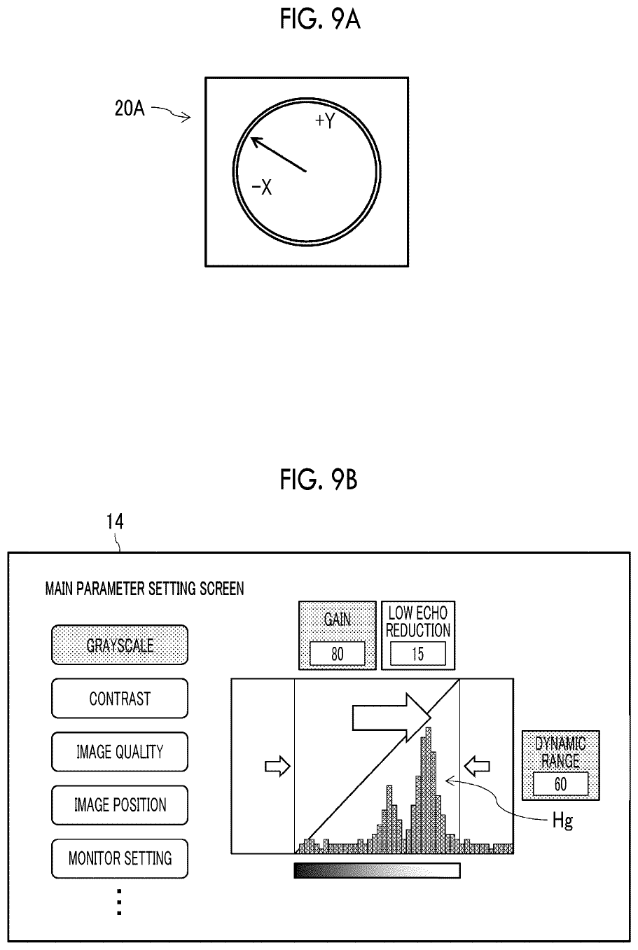

FIG. 9A is a diagram showing a state in which the trackball 20A is operated in the oblique direction between the −X-axis direction and the +Y-axis direction. FIG. 9B is a diagram showing a state in which the value of the main parameter “dynamic range” as the first parameter and the value of the main parameter “gain” as the second parameter have been changed on the main parameter setting screen in response to the operation of the trackball 20A in the oblique direction between the −X-axis direction and the +Y-axis direction. As shown in FIG. 9A, in a case where the trackball 20A is operated in the oblique direction between the −X-axis direction and the +Y-axis direction, the parameter setting unit 40 decreases the value of the main parameter “dynamic range” and increases the value of the main parameter “gain”. This is because, in a case where the operation direction and the operation amount of the trackball 20A shown in FIG. 9A are viewed as a vector, a component in the X-axis direction of the vector is oriented in the −X-axis direction, and a component in the Y-axis direction of the vector is oriented in a +Y-axis direction.

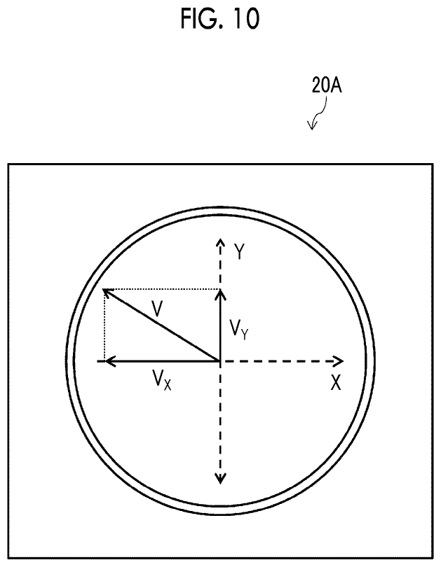

Here, a method of determining the change amounts of the main parameters “dynamic range” and “gain” in a case where the trackball 20A is operated in the oblique direction will be described. FIG. 10 is a diagram showing the operation amount of the trackball 20A in the oblique direction, and the component in the X-axis direction and the component in the Y-axis direction of the operation amount. As shown in FIG. 10, the parameter setting unit 40 decomposes the operation amount of the trackball 20A in the oblique direction (represented by a length of a vector V in FIG. 10) into a component in the X-axis direction (vector V_{X}) and a component in the Y-axis direction (vector V_{Y}). Then, the parameter setting unit 40 determines an amount of change of the main parameter “dynamic range” based on the component in the X-axis direction, that is, the magnitude of the vector V_{X}, and determines an amount of change of the main parameter “gain” based on the component in the Y-axis direction, that is, the magnitude of the vector V_{Y}.

Conceptually, the method of determining the change amounts of the main parameters “dynamic range” and “gain” in a case where the trackball 20A is operated in the oblique direction is as mentioned above. However, in a case where, as in the present embodiment, the sensor 20AcX that detects the operation amount of the trackball 20A in the X-axis direction and the sensor 20AcY that detects the operation amount of the trackball 20A in the Y-axis direction are provided, even in a case where the trackball 20A is operated in the oblique direction, the parameter setting unit 40 need only determine the amount of change of the main parameter “dynamic range” based on the operation amount detected by the sensor 20AcX and determine the amount of change of the main parameter “gain” based on the operation amount detected by the sensor 20AcY.

In a case where the value of the main parameter “dynamic range” decreases and the value of the main parameter “gain” increases, the display control unit 38 changes the graph Gr on the main parameter setting screen such that the left end of the graph Gr moves to the right side (the greater signal intensity side), the right end of the graph Gr moves to the left side (the smaller signal intensity side), and the slope of the graph Gr is greater, and also changes the histogram Hg such that the histogram Hg as a whole moves to the right side (the greater signal intensity side). The changed graph Gr indicates the characteristics of the ultrasound image processed using the changed value of the main parameter “dynamic range”, and the changed histogram Hg indicates the characteristics of the ultrasound image processed using the changed value of the main parameter “gain”.

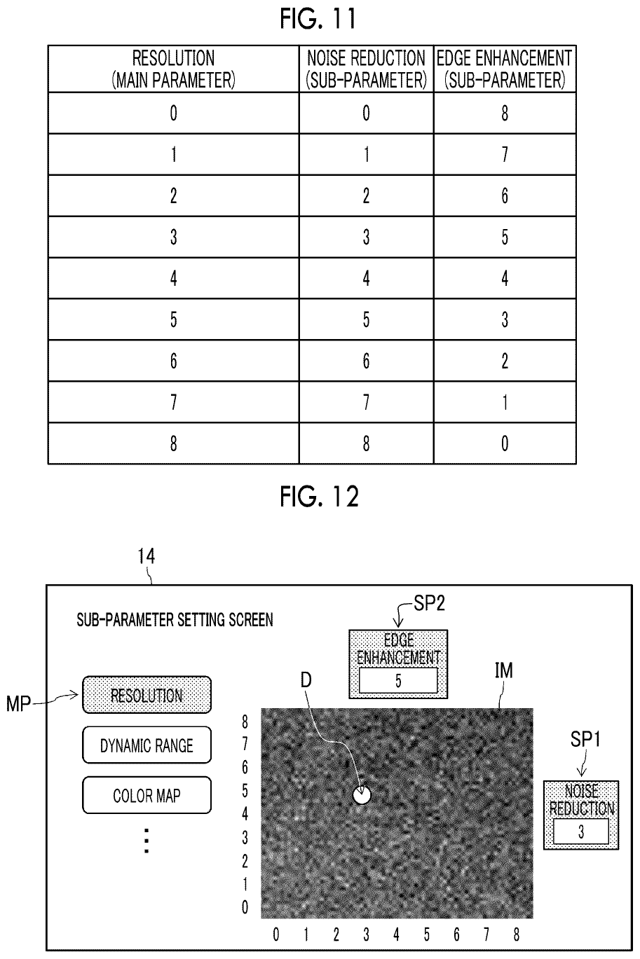

Hereinafter, details of the setting processing of the values of the sub-parameters will be described with reference to FIGS. 11 to 17. First, a relationship between the main parameter and the sub-parameter will be described. As mentioned above, the sub-parameters are positioned subordinate to the main parameter, and a value of a certain main parameter is composed of values of a plurality of sub-parameters. FIG. 11 is a diagram showing examples of the main parameter and the sub-parameters. For example, a value “0” of the main parameter “resolution” is composed of a combination of a value “0” of the sub-parameter “noise reduction” and a value “8” of the sub-parameter “edge enhancement”, and a value “1” of the main parameter “resolution” is composed of a combination of a value “1” of the sub-parameter “noise reduction” and a value “7” of the sub-parameter “edge enhancement”. Setting the values of the plurality of sub-parameters for a certain main parameter means setting the values of the plurality of sub-parameters constituting a certain value of the main parameter. In the present embodiment, the main parameter “resolution”, the sub-parameter “noise reduction”, and the sub-parameter “edge enhancement” are each assumed to be able to take values from “0” to “8”.

In a case where the ultrasound diagnostic apparatus 10 transitions to the sub-parameter setting mode, the display control unit 38 displays the sub-parameter setting screen as shown in FIG. 12 on the display 14. On the sub-parameter setting screen, a plurality of main parameters MP are displayed. The operator selects, from among the plurality of main parameters MP, a main parameter MP whose sub-parameters are to be changed. Here, it is assumed that the main parameter “resolution” is selected.

In a case where the main parameter MP is selected by the administrator or the operator of the ultrasound diagnostic apparatus 10, the display control unit 38 causes the display 14 to display items and values of a plurality of sub-parameters included in the selected main parameter MP. In the example of FIG. 12, the display control unit 38 causes the display 14 to display the item and the value of the sub-parameter “noise reduction” as a first sub-parameter indicated by reference numeral SP1, and an item and a value of the sub-parameter “edge enhancement” as a second sub-parameter indicated by reference numeral SP2.

Preferably, in a case where the main parameter MP is selected by the administrator or the operator of the ultrasound diagnostic apparatus 10, the display control unit 38 may cause the display 14 to display, as the characteristics of the ultrasound image, the characteristic index indicating the characteristics related to the plurality of sub-parameters included in the selected main parameter MP.

In the example of FIG. 12, since the parameter type “resolution” including the sub-parameter “noise reduction” and the sub-parameter “edge enhancement” is selected, the display control unit 38 displays an image IM indicating the resolution of the ultrasound image as the characteristic index. In addition, the display control unit 38 may also regard a display region of the image IM as a graph region in which a horizontal axis represents noise reduction and a vertical axis represents edge enhancement, and display, as the characteristic index, a dot D indicating a position in the graph region corresponding to a current value of the sub-parameter “noise reduction” and a current value of the sub-parameter “edge enhancement”.

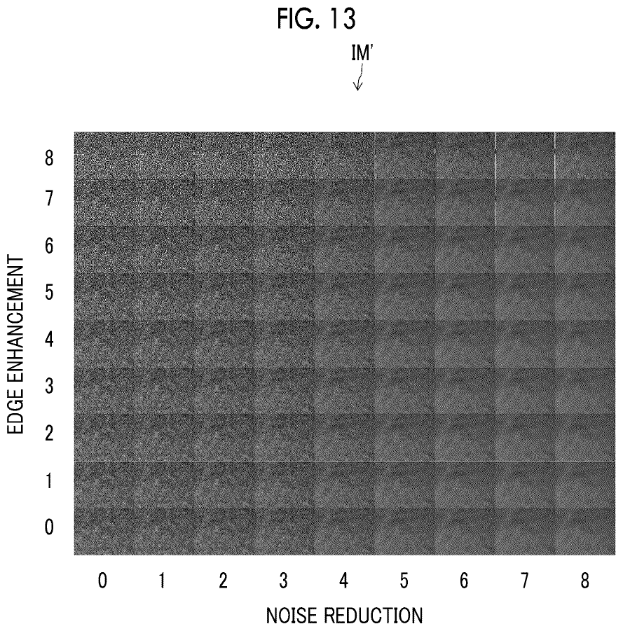

Additionally, as shown in FIG. 13, the characteristic index may be an image IM′ composed of a plurality of images indicating respective resolutions of an ultrasound image, the plurality of images being represented by combinations (9×9=81 types) of values “0” to “8” of the sub-parameter “noise reduction” and values “0” to “8” of the sub-parameter “edge enhancement”.

On the sub-parameter setting screen, the parameter setting unit 40 changes the values of the plurality of sub-parameters included in the selected main parameter MP in response to the operation of the trackball 20A by the administrator or the operator of the ultrasound diagnostic apparatus 10. Specifically, the parameter setting unit 40 determines, according to the operation direction of the trackball 20A, the sub-parameter whose value is to be changed from among the plurality of sub-parameters. In the present embodiment, in a case where the trackball 20A is operated in the first axial direction (the X-axis direction (refer to FIG. 2), that is, the lateral direction), the parameter setting unit 40 changes the value of the sub-parameter “noise reduction” as the first sub-parameter, and in a case where the trackball 20A is operated in the second axial direction (the Y-axis direction (refer to FIG. 2), that is, the longitudinal direction), the parameter setting unit 40 changes the value of the sub-parameter “edge enhancement” as the second sub-parameter.

In a case where the trackball 20A is operated in the X-axis direction (lateral direction), the value of the sub-parameter “noise reduction” is changed, and in a case where the trackball 20A is operated in the Y-axis direction (longitudinal direction), the value of the sub-parameter “edge enhancement” is changed. Accordingly, the display control unit 38 may preferably display an item and a value of the sub-parameter “noise reduction” and an item and a value of the sub-parameter “edge enhancement” such that the administrator or the operator can easily visualize which sub-parameter value is changed depending on the direction in which the trackball 20A is operated. For example, as shown in FIG. 12, the display control unit 38 may preferably display, on the left side or the right side of the characteristic index (the image IM and the dot D), the sub-parameter “noise reduction” whose value is to be changed in a case where the trackball 20A is operated in the X-axis direction (lateral direction), and display, on the upper side or the lower side of the characteristic index, the sub-parameter “edge enhancement” whose value is to be changed in a case where the trackball 20A is operated in the Y-axis direction (longitudinal direction).

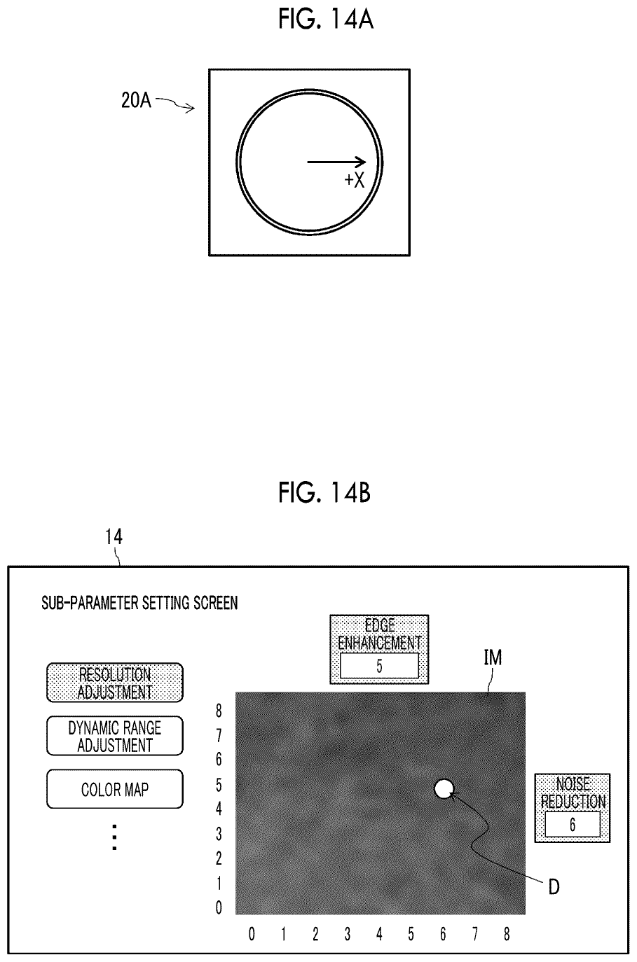

FIG. 14A is a diagram showing a state in which the trackball 20A is operated in the +X-axis direction. FIG. 14B is a diagram showing a state in which the value of the sub-parameter “noise reduction” as the first sub-parameter has been changed on the sub-parameter setting screen in response to the operation of the trackball 20A in the +X-axis direction. As shown in FIG. 14A, in a case where the trackball 20A is operated in the +X-axis direction, the parameter setting unit 40 increases the value of the sub-parameter “noise reduction”. As mentioned above, since the trackball 20A can detect the operation amount (the rotation amount of the ball portion 20Ab), the parameter setting unit 40 may preferably determine the change amount of the value of the sub-parameter “noise reduction” in accordance with the operation amount of the trackball 20A in the +X-axis direction.

In a case where the value of the sub-parameter “noise reduction” increases, the display control unit 38 changes the image IM on the sub-parameter setting screen. The changed image IM shows the characteristics of the ultrasound image processed using the changed value of the sub-parameter “noise reduction”. In addition, the display control unit 38 displays the dot D at a position corresponding to the changed value of the sub-parameter “noise reduction”. That is, the display control unit 38 may preferably cause the display unit to display the characteristic index indicating the characteristics of the ultrasound image processed using the changed sub-parameter. As a result, the administrator or the operator of the ultrasound diagnostic apparatus 10 can easily understand the characteristics of the ultrasound image after the sub-parameter has been changed.

On the contrary, in a case where the trackball 20A is operated in the −X-axis direction, the parameter setting unit 40 decreases the value of the sub-parameter “noise reduction”. Here, the parameter setting unit 40 may preferably determine the change amount of the value of the sub-parameter “noise reduction” in accordance with the operation amount of the trackball 20A in the −X-axis direction.

Even in a case where the value of the sub-parameter “noise reduction” decreases, the display control unit 38 changes the image IM on the sub-parameter setting screen so as to indicate the characteristics of the ultrasound image processed using the changed value of the sub-parameter “noise reduction”, and displays the dot D at a position corresponding to the changed value of the sub-parameter “noise reduction”.

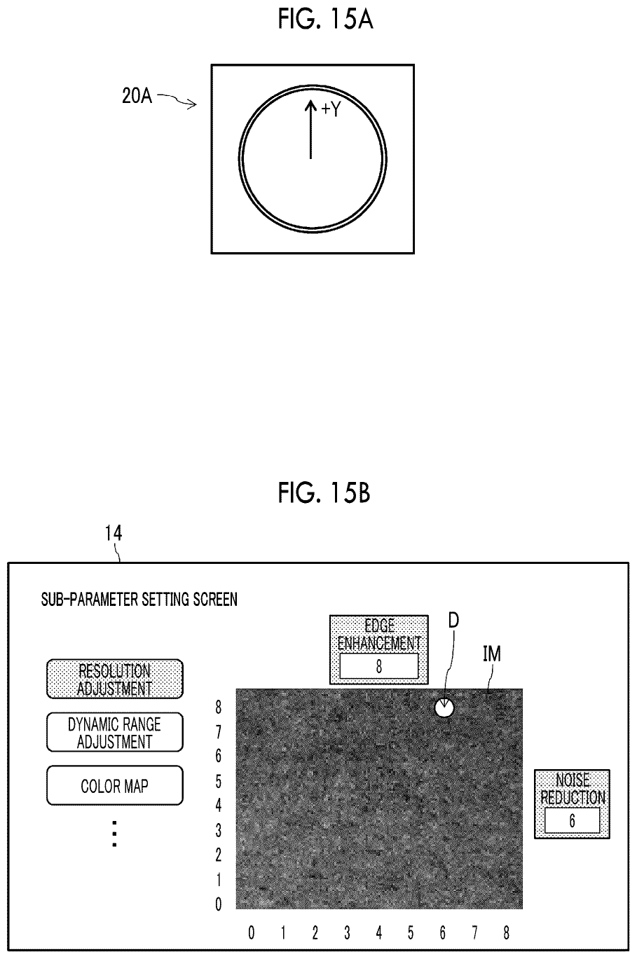

FIG. 15A is a diagram showing a state in which the trackball 20A is operated in the +Y-axis direction. FIG. 15B is a diagram showing a state in which the value of the sub-parameter “edge enhancement” as the second sub-parameter has been changed on the main parameter setting screen in response to the operation of the trackball 20A in the +Y-axis direction. As shown in FIG. 15A, in a case where the trackball 20A is operated in the +Y-axis direction, the parameter setting unit 40 increases the value of the sub-parameter “edge enhancement”. Here, the parameter setting unit 40 may preferably determine the change amount of the value of the sub-parameter “edge enhancement” in accordance with the operation amount of the trackball 20A in the +Y-axis direction.

In a case where the value of the sub-parameter “edge enhancement” increases, the display control unit 38 changes the image IM on the main parameter setting screen so as to indicate the characteristics of the ultrasound image processed using the changed value of the sub-parameter “edge enhancement”, and displays the dot D at a position corresponding to the changed value of the sub-parameter “edge enhancement”.

On the contrary, in a case where the trackball 20A is operated in the −Y-axis direction, the parameter setting unit 40 decreases the value of the sub-parameter “edge enhancement”. Here, the parameter setting unit 40 may preferably determine the change amount of the value of the sub-parameter “edge enhancement” in accordance with the operation amount of the trackball 20A in the −Y-axis direction.

In a case where the value of the sub-parameter “edge enhancement” decreases, the display control unit 38 changes the image IM on the main parameter setting screen so as to indicate the characteristics of the ultrasound image processed using the changed value of the sub-parameter “edge enhancement”, and displays the dot D at a position corresponding to the changed value of the sub-parameter “edge enhancement”.

In this manner, according to the present embodiment, the administrator or the operator of the ultrasound diagnostic apparatus 10 can change the values of the two sub-parameters only by operating the trackball 20A. Accordingly, the administrator or the operator of the ultrasound diagnostic apparatus 10 can easily change the values of the plurality of sub-parameters related to the ultrasound image processing as compared with the conventional case.

Similarly to a case where the main parameter is set, the parameter setting unit 40 may switch the sub-parameter whose value is to be changed in a case where the trackball 20A is operated in response to the operation of the operation button 20B. In addition, similarly to the main parameter setting screen, the display control unit 38 may preferably explicitly indicate on the sub-parameter setting screen which sub-parameter is to have its value changed by the operation of the trackball 20A.

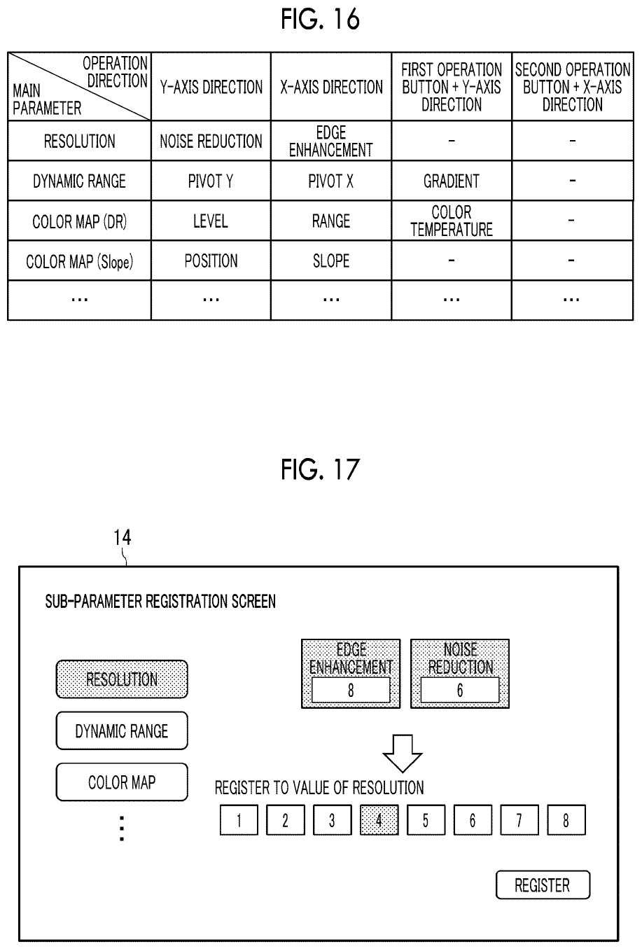

Which sub-parameter is to be changed in a case where the trackball 20A is operated in each of the X-axis direction and the Y-axis direction or in a case where the trackball 20A is operated in each of the X-axis direction and the Y-axis direction while the operation button 20B is being pressed is set in advance. FIG. 16 is a diagram showing a correspondence relationship between the operation direction of the trackball 20A and the presence or absence of the operation of the operation button 20B, and the sub-parameter to be changed for each main parameter MP.

In the table shown in FIG. 16, each row corresponds to each main parameter MP, and each column corresponds to the operation content of the input interface 20. For example, with respect to the main parameter “resolution”, the sub-parameter “edge enhancement” is set for the operation of the trackball 20A in the Y-axis direction, and the sub-parameter “noise reduction” is set for the operation of the trackball 20A in the X-axis direction. Additionally, with respect to the main parameter “dynamic range”, the sub-parameter “pivot Y” is set for the operation of the trackball 20A in the Y-axis direction, the sub-parameter “pivot X” is set for the operation of the trackball 20A in the X-axis direction, and the sub-parameter “gradient” is set for the operation of the first operation button 20B and the trackball 20A in the Y-axis direction.

The table as shown in FIG. 16 is stored in the memory 18. The parameter setting unit 40 determines the sub-parameter whose value is to be changed in a case where the trackball 20A is operated, or in a case where the operation button 20B and the trackball 20A are operated, in accordance with the main parameter MP selected by the operator, while referring to the table.

In addition, in the example of FIG. 12, similarly to a case where the main parameter is set, the parameter setting unit 40 may preferably change both the value of the sub-parameter “noise reduction” as the first sub-parameter and the value of the sub-parameter “edge enhancement” as the second sub-parameter in a case where the trackball 20A is operated in the oblique direction.

FIG. 17 is a diagram showing an example of a registration screen of the sub-parameter. After the setting of values of the plurality of sub-parameters has been completed, the display control unit 38 displays a sub-parameter registration screen, such as that shown in FIG. 17, in accordance with an instruction from the administrator or the operator of the ultrasound diagnostic apparatus 10. The sub-parameter registration screen is a screen for determining with which value of a corresponding main parameter the values of the plurality of sub-parameters that have been set are to be registered.

For example, in the example of FIG. 17, the value of the sub-parameter “noise reduction” is set to “6”, and the value of the sub-parameter “edge enhancement” is set to “8”. In the sub-parameter registration screen, in a case where the value “4” of the main parameter “resolution” is selected by the administrator or the operator of the ultrasound diagnostic apparatus 10 and the registration button is operated, the parameter setting unit 40 registers the value “6” of the sub-parameter “noise reduction” and the value “8” of the sub-parameter “edge enhancement” as values constituting the value “4” of the main parameter “resolution”.

Although the ultrasound image processing apparatus according to the present disclosure has been described above, the ultrasound image processing apparatus according to the present disclosure is not limited to the embodiment described above, and various modifications can be made without departing from the gist of the present disclosure.

For example, in the embodiment described above, the ultrasound image processing apparatus is the ultrasound diagnostic apparatus 10, but the ultrasound image processing apparatus may be an apparatus other than the ultrasound diagnostic apparatus 10. For example, the ultrasound image processing apparatus may be another apparatus such as a server computer. In addition, each of the above-mentioned processes executed by the ultrasound image processing apparatus may be executed in a distributed manner in a plurality of apparatuses.

In the present embodiment, each process is executed by any computer. Additionally, any computer may execute these processes by means of a processor serving as hardware, a program serving as software, or a combination of these. In that case, the processor is configured to execute various kinds of processes in the present embodiment in cooperation with the program, and can function as each unit or each means in the present embodiment. Further, the execution order of the processes by the processor is not limited to the order described above and may be changed as appropriate. Any computer may be a general-purpose computer, a computer for a specific application, a workstation, or another system capable of executing each process.

The processor may be configured using one or a plurality of pieces of hardware, and the type of hardware is not limited. For example, the processor can be configured using hardware, such as a central processing unit (CPU), a micro processing unit (MPU), a programmable logic device, such as a field programmable gate array (FPGA), a dedicated circuit for executing specific processing, such as an application specific integrated circuit (ASIC), a graphic processing unit (GPU), or a neural processing unit (NPU). In addition, the type of hardware may be a combination of different types of hardware. In a case where a plurality of pieces of hardware are configured to execute one or a plurality of processes of a certain processor, the plurality of pieces of hardware may be present in physically separate devices or may be present in the same device. Further, in any embodiment, the order of the processes by the processor is not limited to the order described above and may be changed as appropriate. It should be noted that the hardware is configured with an electrical circuit (circuitry) formed by combining circuit elements such as semiconductor elements.

Furthermore, the program may be software, such as firmware or a microcode. Moreover, the program may be, for example, a program module group, and each function thereof may be implemented by a processor configured to execute the corresponding function. The program may be a program code or a plurality of code segments stored in one or a plurality of non-transitory computer-readable media (for example, a storage medium, other storages, or the like). The program may be stored in a distributed manner across a plurality of non-transitory computer-readable media present in physically separated devices. The program code or code segments may represent any combination of procedures, functions, subprograms, routines, subroutines, modules, software packages, classes, or commands, data structures, or program statements. The program code or code segments may be connected to other code segments or hardware circuits by transmitting and receiving information, data, arguments, parameters, or contents of a memory.

The present invention is also applicable to a program and a program product.

What is claimed is:

1. An ultrasound image processing apparatus comprising:
a processor; and
a pointing device that is operable by an operator in a plurality of directions,
wherein the processor is configured to:
in a case where the pointing device is operated in a first axial direction, change a value of a first parameter, which is a parameter related to ultrasound image processing that is processing relating to an ultrasound image formed by transmitting and receiving an ultrasound wave with respect to a subject; and
in a case where the pointing device is operated in a second axial direction different from the first axial direction, change a value of a second parameter, which is a parameter related to the ultrasound image processing and different from the first parameter.

2. The ultrasound image processing apparatus according to claim 1,
wherein the processor is configured to:
change the values of the first parameter and the second parameter in a case where the pointing device is operated in a third axial direction that is a direction between the first axial direction and the second axial direction.

3. The ultrasound image processing apparatus according to claim 2,
wherein the pointing device is configured to enable the operator to input an operation amount in each direction, and
the processor is configured to:
determine a change amount of the value of the first parameter based on a component in the first axial direction of an operation amount in the third axial direction; and
determine a change amount of the value of the second parameter based on a component in the second axial direction of the operation amount in the third axial direction.

4. The ultrasound image processing apparatus according to claim 1,
wherein the parameter includes a main parameter and a plurality of sub-parameters for determining a value of the main parameter,
the ultrasound image processing apparatus is operable in a first operation mode in which values of a plurality of the main parameters are changeable, and a second operation mode in which values of the plurality of sub-parameters are changeable, and
the processor is configured to:
in a case where the pointing device is operated in the first axial direction while operating in the second operation mode, change a value of a first sub-parameter, which is one of the plurality of sub-parameters; and
in a case where the pointing device is operated in the second axial direction, change a value of a second sub-parameter, which is a sub-parameter different from the first sub-parameter.

5. The ultrasound image processing apparatus according to claim 1,
wherein the processor is configured to:
cause a display unit to display a characteristic index indicating characteristics of an ultrasound image processed using the changed first parameter and the changed second parameter.

6. A non-transitory computer-readable storage medium storing an ultrasound image processing program for causing a computer including a pointing device that is operable by an operator in a plurality of directions to execute:
in a case where the pointing device is operated in a first axial direction, changing a value of a first parameter, which is a parameter related to ultrasound image processing that is processing relating to an ultrasound image formed by transmitting and receiving an ultrasound wave with respect to a subject; and
in a case where the pointing device is operated in a second axial direction different from the first axial direction, changing a value of a second parameter, which is a parameter related to the ultrasound image processing and different from the first parameter.
